SEQUENCE LISTING (9 sequences)

SEQ ID NO: 1 - protein, 548 aa, Homo sapiens
MKTLETQPLAPDCCPSDQDPAPAHPSPHASPMNKNADSELMPPPPERGDPPRLSPDPVAGSAVSQELREGDPVSLSTPLETEFGSPSELSPRIEEQELSENTSLPAEEANGSLSEEEANGPELGSGKAMEDTSGEPAAEDEGDTAWNYSFSQLPRFLSGSWSEFSTQPENFLKGCKWAPDGSCILTNSADNILRIYNLPPELYHEGEQVEYAEMVPVLRMVEGDTIYDYCWYSLMSSAQPDTSYVASSSRENPIHIWDAFTGELRASFRAYNHLDELTAAHSLCFSPDGSQLFCGFNRTVRVFSTARPGRDCEVRATFAKKQGQSGIISCIAFSPAQPLYACGSYGRSLGLYAWDDGSPLALLGGHQGGITHLCFHPDGNRFFSGARKDAELLCWDLRQSGYPLWSLGREVTTNQRIYFDLDPTGQFLVSGSTSGAVSVWDTDGPGNDGKPEPVLSFLPQKDCTNGVSLHPSLPLLATASGQRVFPEPTESGDEGEELGLPLLSTRHVHLECRLQLWWCGGAPDSSIPDDHQGEKGQGGTEGGVGELI

SEQ ID NO: 2 - DNA, 1982 bp, Homo sapiens
gtcccggctccgcgggttccgtgggtcgcccgcgaaatctgatccgggatgcggcggcccaatcggaaggtggaccgaaatcccgcgacagcaagaggcccgtagcgacccgcggtgctaaggaacacagtgctttcaaaagaattggcgtccgctgttcgcctctcctcccgggagtcttctgcctactcccagaagaggagggaagcacaggtgggtttctttagctctgcgtcggatccctgagaacttcgaagccatcctggctgaggctaatctccgctgtgcttcctctgcagtatgaagactttggagactcaaccgttagctccggactgctgtccttcagaccaggacccagctccagcccatccttctccccacgcttccccgatgaataaaaatgcggactctgaactgatgccaccgcctcccgaaaggggggatccgccccggttgtccccagatcctgtggctggctcagctgtgtcccaggagctacgggagggggacccagtttctctctccactcccctggaaacagagtttggttcccctagtgagttgagtcctcgaatcgaggagcaagaactttctgaaaatacaagccttcctgcagaagaagcaaacgggagcctttctgaagaagaagcgaacgggccagagttggggtctggaaaagccatggaagatacctctggggaacccgctgcagaggacgagggagacaccgcttggaactacagcttctcccagctgcctcgatttctcagtggttcctggtcagagttcagcacccaacctgagaacttcttgaaaggctgtaagtgggctcctgacggttcctgcatcttgaccaatagtgctgataacatcttgcgaatttataacctgcccccagagctgtaccatgagggggagcaggtggaatatgcagaaatggtccctgtccttcgaatggtggaaggtgataccatctatgattactgctggtattctctgatgtcctcagcccagccagacacctcctacgtggccagcagcagccgggagaacccgattcatatctgggacgcattcactggagagctccgggcttcctttcgcgcctacaaccacctggatgagctgacggcagcccattcgctctgcttctccccggatggctcccagctcttctgtggcttcaaccggactgtgcgtgttttttccacggcccggcctggccgagactgcgaggtccgagccacatttgcaaaaaagcagggccagagcggcatcatctcctgcatagccttcagcccagcccagcccctctatgcctgtggctcctacggccgctccctgggtctgtatgcctgggatgatggctcccctctcgccttgctgggagggcaccaagggggcatcacccacctctgctttcatcccgatggcaaccgcttcttctcaggagcccgcaaggatgctgagctcctgtgctgggatctccggcagtctggttacccactgtggtccctgggtcgagaggtgaccaccaatcagcgcatctacttcgatctggacccgaccgggcagttcctagtgagtggcagcacgagcggggctgtctctgtgtgggacacggacgggcctggcaatgatgggaagccggagcccgtgttgagttttctgccccagaaggactgcaccaatggcgtgagcctgcaccctagcctgcctctcctggccactgcctccggtcagcgtgtgtttcctgagcccacagagagtggggacgaaggagaggagctgggccttcccttgctctccacgcgccacgtccaccttgaatgtcggcttcagctctggtggtgtgggggggcgccagactccagcatccctgatgatcaccagggcgagaaagggcagggaggaacggagggaggtgtgggtgagctgatataaaaaggtttttatgataaaaaaaaaaaaaaaaaaaa

SEQ ID NO: 3 - DNA, 8 bp, Artificial Sequence
  Oligonucleotide primer
agtcagtc

SEQ ID NO: 4 - DNA, 8 bp, Artificial Sequence
  Oligonucleotide primer
aatcaatc

SEQ ID NO: 5 - DNA, 6 bp, Artificial Sequence
  Oligonucleotide primer
agtgtc

SEQ ID NO: 6 - protein, 14 aa, Artificial Sequence
  Peptide immunogen
RVFPEPTESGDEGE

SEQ ID NO: 7 - protein, 15 aa, Artificial Sequence
  Peptide immunogen
GKPEPVLSFLPQKDC

SEQ ID NO: 8 - DNA, 21 bp, Artificial Sequence
  Inhibitory oligonucleotide
ccgggagaacccgattcatat

SEQ ID NO: 9 - DNA, 21 bp, Artificial Sequence
  Inhibitory oligonucleotide
aacgggagcctttctgaagaa

(19) United States
(12) Patent Application Publication
(10) Pub. No.: US 2011/0097732 A1
(43) Pub. Date: Apr. 28, 2011

(54) NOVEL BIOMARKER FOR THE PROGNOSIS OF BREAST CANCER

(75) Inventors: Anita Langerød, Oslo (NO); Marianne Farnebo, Stockholm (SE)

(73) Assignee: MEDINNOVA AS

(21) Appl. No.: 12/911,656

(22) Filed: Oct. 25, 2010

Related U.S. Application Data
(60) Provisional application No. 61/254,884, filed on Oct. 26, 2009.

(51) Int. Cl. G01N 33/574; C07H 21/04; C07K 16/18

(52) U.S. Cl. 435/7.1; 536/23.53; 530/387.9; 530/387.3

(57) ABSTRACT

The present invention relates to methods of determining the predilection for survival for an individual having breast cancer comprising: obtaining a breast tissue sample from said individual, measuring the amount of Wrap53 in the nucleus of cells in said breast tissue sample, and/or measuring the amount of Wrap53 in the cytoplasm of cells in said breast tissue sample, and wherein both the nuclear levels, the cytoplasmic levels and/or the ratio between the nuclear and cytoplasmic levels of Wrap53 may be used alone or in combination in breast cancer prognosis. The invention furthermore relates to antibodies and kits relating to Wrap53.

### CROSS-REFERENCE TO RELATED APPLICATIONS

This application is a non-provisional of and claims the benefit of priority to U.S. provisional Patent Application Ser. No. 61/254,884, filed on Oct. 26, 2009, the disclosure of which is hereby expressly incorporated by reference in its entirety.

### SEQUENCE LISTING

The present application is being filed along with a Sequence Listing in electronic format. The Sequence Listing is provided as a file entitled SequenceListing.TXT, created Oct. 25, 2010, which is 8.49 Kb in size. The information in the electronic format of the Sequence Listing is incorporated herein by reference in its entirety.

### FIELD OF THE INVENTION

The present invention relates to the use of the Wrap53 protein as a biomarker for breast cancer prognosis. In particular, some embodiments relate to the use of the Wrap53 protein as a biomarker for breast cancer prognosis, wherein in a low level of Wrap53 in the nucleus and/or a low ratio between the level of Wrap53 in the nucleus to the level of Wrap53 in the cytoplasm is indicative of poor prognosis for breast cancer.

### DESCRIPTION OF THE RELATED ART

Breast cancer is the leading cause of death among women between 40 and 55 years of age and is the second overall cause of death among women (source: the American Cancer Society). Recent progress in mass screening programs, diagnostics, and adjuvant systemic therapies has resulted in increased survival and declined mortality. Nevertheless, the incidence is significantly increasing and one of four patients experience recurrence and die from metastatic disease.

Currently, the choice of treatment for breast cancer patients largely depends on only a handful of clinical-, histopatholocical- and molecular markers, used to determine the prognosis of the patient as well as to determine the likelihood of the patients to respond to certain treatments. Although, the mortality rate from breast cancer has decreased in recent years a large number of women are not cured and new more exact markers are needed to better predict and identify the high risk group with poor survivors. By identifying this high risk group with poor survival rate from the low risk group, altered treatment strategies will improve the clinical outcome, including less adverse side effects and increased lifespan. New biomarkers and breast cancer assays are needed to avoid over-treatment of patients with low risk and under-treatment of high-risk patients, as well as, to provide personalized and targeted treatment.

### Wrap53

Wrap53 is a natural p53 antisense transcript that interacts with the 5′UTR of p53 and so controls p53 induction upon DNA damage. Wrap53 is located on chromosome 17p13 and overlaps the p53 gene in a head-to-head fashion. The gene has three alternative start exons (1α, 1β and 1γ) and exon 1α directly overlaps the first exon of p53 in an antisense fashion. Wrap53 also encodes a protein homologous to members of the WD40 protein family, hence the name Wrap53. This is in contrast to many other regulatory RNAs that are non-coding and exert their function only at the RNA level. The Wrap53 protein (also denoted WDR79 and TCAB1) was recently identified as a Cajal body protein that binds and directs small Cajal body-specific RNAs (scaRNAs), including telomerase RNA, to Cajal bodies (Farnebo M. Wrap53, a novel regulator of p53. Cell Cycle. 2009 Aug;8(15):2343-6).

Montserrat Garcia-Closas et al., present a case control study investigating the risk of breast cancer based on “normal” variation in the genome. Variation in the DNA from normal cells (extracted from blood) are analysed in women with and without breast cancer. The paper discloses that genetic variation in the WDR79 gene (Wrap53) may increase the susceptibility to breast cancer. WO 2008/041933 A2 discloses a small nucleic acid molecule that down-regulates expression of Wrap53 gene via RNA interference for the treatment of hyperproliferative disorders, such as cancer. Similar to the findings presented in WO 2008/041933 A2, the Wrap53 protein/RNA has also been reported to be upregulated in brain tumors (Zhang, Bioinformatics, (20) p 2390-8, 10 2004). See specifically Supplementary Table 1, where Wrap53 is listed as NM 018081 =NCBI GenBank number). Despite these advances, the need for biomarkers and prognosis methods for breast cancer are manifest.

### SUMMARY OF THE INVENTION

In the present application the inventors have surprisingly discovered that an up-regulation (compared to a reference level) of Wrap53 protein in the nucleus is indicative of a better prognosis for breast cancer patients. Similar, it has been discovered that an increased ratio between the level or amount of Wrap53 in the nucleus to the level or amount of Wrap53 in the cytoplasm (compared to a reference level) is indicative of a better prognosis for breast cancer patients.

In some contexts, the term “upregulated” is used herein to refer to an amount that is above a reference level or base value, which may be simply the mere presence of the molecule when the reference level or base value is zero or no detectable amount. That is, for example, “upregulated” may be an amount or level that is at least, equal to, or greater than about 0.1-0.5, 0.5-1.0, 1.0-1.3, 1.3-1.5, 1.5-2.0, 3.0, 4.0, 5.0 fold greater amount than a reference or base level (for example a baseline measurement made with a control sample) or at least, equal to, or greater than any number in the aforementioned ranges.

Thus, some embodiments concern the use of Wrap53 as a biomarker in breast cancer. In particular, some embodiments provide Wrap53 as a prognostic marker that solves the above mentioned problems of the prior art with predicting and identifying the high risk group with poor survivors and avoid over-treatment of patients with low risk and under-treatment of high-risk patients.

The present invention relates to methods where a breast cancer sample is analysed for Wrap53 levels. Preferably the analysis is performed using immunohistochemical assays. The sample is then analysed for levels of Wrap53 in the nucleus and in the cytoplasm. An indication of better survival is present when (compared to a reference or base level) an up-regulation in the nucleus of Wrap53 is present, a down-regulation in the cytoplasm is present or where an increased ratio of nuclear Wrap53 levels to cytoplasmic Wrap53.

Thus, one aspect of the present invention relates to a method for determining the prognosis of breast cancer comprising:
measuring the level of Wrap53 in a biological sample obtained from an individual,
comparing said level with a reference level, and
determining that the individual is likely to have a poor prognosis when:
1) the level of Wrap53 in the nucleus of cells in the sample is below the reference level
2) the ratio between the level of Wrap53 in the nucleus of cells in the sample to the level of Wrap53 in the cytoplasm of cells in the sample is below the reference level and/or
3) the level of Wrap53 in the cytoplasm of cells in the sample is above the reference level; or
determining that the individual is likely to have a good prognosis of breast cancer when:
1) the level of Wrap53 in the nucleus of cells in the sample is above or equal to the reference level,
2) the ratio between the level of Wrap53 in the nucleus of cells in the sample and the level of Wrap53 in the cytoplasm of cells in the sample is above or equal to the reference level, and/or
3) the level of Wrap53 in the cytoplasm of cells in the sample is below or equal to the reference level.

Thus, both the nuclear levels, the cytoplasmic levels and/or the ratio between the nuclear and cytoplasmic levels of Wrap53 may be used alone or in the different combinations in breast cancer prognosis.

### Wrap53 and ROC-Curves

In some embodiments, ROC-curves may be used to establish specificity and sensitivity of an assay. Thus, in another aspect the invention relates to method for determining the prognosis of breast cancer comprising:
a) measuring the level of Wrap53 in a biological sample obtained from an individual,
b) comparing said level with a reference level,
c) generating a ROC curve containing values representing the level of Wrap53 determined in the biological sample and the reference level,
d) selecting a desired sensitivity,
e) determining the specificity corresponding to the desired sensitivity from the ROC curve,
f) determining that the individual is likely to have a poor prognosis when:
   1) the level of Wrap53 in the nucleus of cells in the sample is below the reference level corresponding to the desired specificity,
   2) the ratio between the Wrap53 in the nucleus of cells in the sample and the level of Wrap53 in the cytoplasm of cells in the sample is below the reference level corresponding to the desired specificity, and/or
   3) the level of Wrap53 in the cytoplasm of cells in the sample is above the reference level corresponding to the desired specificity; or
g) determining that the individual is likely to have a good prognosis of breast cancer when:
   1) the level of Wrap53 in the nucleus of cells in the sample is above or equal to the reference level corresponding to the desired specificity,
   2) the ratio between the Wrap53 in the nucleus of cells in the sample and the level of Wrap53 in the cytoplasm of cells in the sample is above or equal to the reference level corresponding to the desired specificity, and/or
   3) the level of Wrap53 in the cytoplasm of cells in the sample is below or equal to the reference level corresponding to the desired specificity.

By using ROC-curves, the skilled person would be able to establish specificity and sensitivity of the assay to a level he finds suitable in the present case.

Thus, both the nuclear levels, the cytoplasmic levels and/or the ratio between the nuclear and cytoplasmic levels of Wrap53 may be used alone or in the different combinations in breast cancer prognosis.

### Determining the Predilection of a Breast Cancer

Similarly, some embodiments concern a method for determining the predilection of a breast cancer. For example, some embodiments concern a method for determining the predilection for survival of an individual with breast cancer comprising:
measuring, at a first time, the level of Wrap53 in a biological sample obtained from an individual that has breast cancer,
measuring, at a second time, the level of Wrap53 in a biological sample obtained from said individual,
comparing the level of Wrap53 measured at said first time to the level of Wrap53 measured at said second time, and
determining the predilection for survival of said individual, wherein:
   1) an increase in the level of Wrap53 in the nucleus of cells in the sample is indicative of an improved survival rate,
   2) a decrease in the level of Wrap53 in the nucleus of cells in the sample is indicative of a reduced survival rate,
   3) an unchanged level of Wrap53 in the nucleus of cells in the sample is indicative of an unchanged survival rate,
   4) an increase in the ratio between the level of Wrap53 in the nucleus of cells in the sample and the level of Wrap53 in the cytoplasm of cells in the sample is indicative of an improved survival rate,
   5) a decrease in the ratio between the level of Wrap53 in the nucleus of cells in the sample and the level of Wrap53 in the cytoplasm of cells in the sample is indicative of a reduced survival rate,
   6) an unchanged ratio between the level of Wrap53 in the nucleus of cells in the sample and the level of Wrap53 in the cytoplasm of cells in the sample is indicative of an unchanged survival rate,
   7) an increase in the level of Wrap53 in the cytoplasm of cells in the sample is indicative of a reduced survival rate,
   8) a decrease in the level of Wrap53 in the cytoplasm of cells in the sample is indicative of an improved survival rate, or
   9) an unchanged level of Wrap53 in the cytoplasm of cells in the sample is indicative of an unchanged survival rate.

By obtaining data at more than one time point, the development of a breast cancer can be determined with respect to Wrap53. Thus, both the nuclear levels, the cytoplasmic levels and/or the ratio between the nuclear and cytoplasmic levels of Wrap53 may be used alone or in the different combinations in the determination of breast cancer development or predilection.

### Antibodies

Robust epitopes and antigenic sequences on Wrap53 have also been discovered and antibodies and binding fragments thereof, which are directed to these epitopes and antigenic sequences are embodiments. Accordingly, some embodiments concern an isolated polynucleotide comprising a nucleic acid sequence encoding a polypeptide capable of specifically binding an exposed epitope on Wrap53 or antigenic sequence thereof, wherein said epitope or antigenic sequence consists of, consists essentially of or comprises at least a portion of position 483-496 or 449-463 of SEQ ID NO.1. For example, some embodiments include an antibody or binding fragment thereof that specifically binds to a polypeptide that consists of, consists essentially of, or comprises positions 483-496 or 449-463 of SEQ ID NO.1. Similarly, some embodiments concern an isolated polypeptide, such as a ligand, capable of specifically binding to an exposed epitope on Wrap53, wherein said epitope consists of, consists essentially of or comprises at least part of position 483-496 or 449-463 of SEQ ID NO.1.

By consisting essentially of, in this context, it is meant to refer to a polypeptide sequence that consists of positions 483-496 or 449-463 of SEQ ID NO.1 and said polypeptide may have at least, less than, or equal to one, two, three, four, five, six, seven, or eight amino acid changes so long as the amino acid change does not prevent the binding of an antibody that is specific for a polypeptide consisting of a sequence of positions 483-496 or 449-463 of SEQ ID NO.1.

The desired polypeptide may be expressed by a nucleic acid construct in vitro or in vivo. Thus, in some embodiments, a nucleic acid construct comprising an isolated polynucleotide as described herein, and a promoter for directing an expression of said isolated polynucleotide in cells is contemplated. The cells may be eukaryotic, preferably mammalian, prokaryotic, bacterial, insect, or yeast.

### Diagnostic Kit

Still more embodiments concern a kit comprising an antibody directed to the specific epitopes described herein. Thus, some embodiments concern a kit for indicating a breast cancer prognosis or determining the course of a breast cancer comprising:
an antibody against at least part of position 483-496 or 449-463 of Wrap53 (SEQ ID NO.1),
an instruction manual explaining how to carry out at least one of the methods according to the preceding claims.

A diagnostic kit as described above can be important for improving diagnosis/prognosis for breast cancer patients.

### Use of Wrap53 as a Biomarker in Breast Cancer

Some embodiments concern methods of using WRAP53 as a biomarker for breast cancer. By some approaches, said methods include a method of determining the predilection for survival for an individual having breast cancer comprising:
obtaining a breast tissue sample from said individual,
   measuring the amount of Wrap53 in the nucleus of cells in said breast tissue sample,
   measuring the amount of Wrap53 in the cytoplasm of cells in said breast tissue sample,
   comparing the amount Wrap53 measured in the nucleus of cells in said breast tissue sample to the amount of Wrap53 measured in the cytoplasm of cells in said breast tissue sample, and
   determining the predilection for survival for said individual having breast cancer.

### Method of Evaluating a Treatment Protocol for an Individual Having Breast Cancer

Wrap53 may also be used to evaluate a treatment protocol for breast cancer patients. Thus, some embodiments concern a method of evaluating a treatment protocol for an individual having breast cancer comprising:
obtaining a first breast tissue sample from an individual having breast cancer prior to initiating a treatment protocol for breast cancer,
measuring the amount of Wrap53 in the nucleus of cells in said first breast tissue sample,
measuring the amount of Wrap53 in the cytoplasm of cells in said first breast tissue sample,
comparing the amount Wrap53 measured in the nucleus of cells in said first breast tissue sample to the amount of Wrap53 measured in the cytoplasm of cells in said first breast tissue sample,
obtaining a second breast tissue sample from said individual after initiation or completion of said treatment protocol for breast cancer,
measuring the amount of Wrap53 in the nucleus of cells in said second breast tissue sample,
measuring the amount of Wrap53 in the cytoplasm of cells in said second breast tissue sample,
comparing the amount Wrap53 measured in the nucleus of cells in said second breast tissue sample to the amount of Wrap53 measured in the cytoplasm of cells in said second breast tissue sample, and
determining the progress of said treatment protocol by comparing the amounts of Wrap53 measured in the nucleus and/or cytoplasm of cells in said second breast tissue sample to the amount of Wrap53 measured in the nucleus and/or cytoplasm of cells in said first breast tissue sample.

### BRIEF DESCRIPTION OF THE FIGURES

FIG. 1 shows the peptide sequences targeted by Wrap53 (483) and Wrap53 (449) antibodies.

FIG. 2 shows Western blot analysis of Wrap53. (A) Western blot analysis of Wrap53 knockdown and Wrap53 overexpression using the Wrap53 (483) antibody. U2OS cells were treated with siWrap53 oligos (siWrap53#1-2) for 48h or CMV-Wrap53 plasmid for 24 hours. β-actin was used as loading control.

(B) Western blot analysis of Wrap53 knockdown and Wrap53 overexpression using the Wrap53 (449) antibody. U2OS cells were treated with siWrap53#2 oligo for 48h or CMV-Wrap53 plasmid for 24 hours.

FIG. 3 shows Immunofluorescence staining of endogenous Wrap53 protein. (A) Immunofluorescence staining of endogenous Wrap53 protein in MCF-7 cells using a Wrap53 specific antibody. (B) Immunofluorescence staining of endogenous Wrap53 protein in human diploid fibroblasts (HDF) cells using a Wrap53 specific antibody. Nuclei were stained with DAPI in both experiments. White arrows indicate Cajal bodies.

FIG. 4 shows that IHC staining of the Wrap53 protein in breast carcinomas showed diverse patterns. (A) Positive nucleus, negative cytoplasm; (B): Positive nucleus, positive cytoplasm; (C): Negative nucleus, positive cytoplasm; D: Negative nucleus, negative cytoplasm.

FIG. 5 shows a Kaplan-Meier plot of breast cancer related survival in patients with or without nuclear staining in combination with or without cytoplasmic staining of the Wrap53 protein. The p-value from the log rank test is shown in the panel (n=170).

FIG. 6A and B show Kaplan-Meier survival curves demonstrating breast cancer patients (test series) with or without nuclear Wrap53 staining, stratified for TP53 mutation status, i. e. wild type (wt) and mutant (mut) TP53. The p-value from log rank test is shown in each panel.

FIG. 7 shows a survival analysis using Cox regression model. Presence/absence of nuclear WRAP53 protein is a strong prognostic factor both in univariate and multivariate survival analysis.

FIG. 8 shows IHC staining of the WRAP53 protein in two breast carcinomas using two different polyclonal antibodies; WRAP53 (483) antibody, and commercial WRAP53 (1-50) antibody. Patient A: Some cells with positive nuclear stain is seen using WRAP53 (483), whereas no cells with positive nuclear stain is seen using

WRAP53 (1-50). Patient B: Strong positive nuclear staining and diffuse cytoplasmic staining is seen using WRAP53 antibody (483), whereas the WRAP53 antibody (1-50) display strong cytoplasmic staining and positive nuclear stain.

### DETAILED DESCRIPTION OF THE INVENTION

### Definitions

The following terms and conventions will first be used throughout this disclosure:

ROC curve

ROC curve: The accuracy of a diagnostic test is characterized by a Receiver Operating Characteristic curve (“ROC curve”). An ROC is a plot of the true positive rate against the false positive rate for the different possible cut points of a diagnostic test. An ROC curve shows the relationship between sensitivity and specificity. That is, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect/test, while an area of 0.5 represents a less useful test. Thus, biomarker and diagnostic methods of the present invention have an AUC greater than 0.50, more preferred tests have an AUC greater than 0.60, more preferred tests have an AUC greater than 0.70.

Other useful measures of the utility of a test are positive predictive value and negative predictive value. Positive predictive value is the percentage of people who test positive that are actually positive. Negative predictive value is the percentage of people who test negative that are actually negative.

### Neoplastic Disorder

Neoplastic disorder can be a disorder comprising an abnormal mass of tissue or cells as a result of neoplasia. Neoplasia is the abnormal proliferation of cells. Neoplasms may be benign, pre-malignant or malignant. The term solid tumour is synonymous with a neoplasm that has formed a lump. Some neoplastic disorders do not cause a lump; they include haematological leukaemia and non-solid tumours such as most forms of carcinoma in situ. Cancer is a malignant neoplastic disorder.

### Operably Linked

The term “operably linked” refers to the connection of elements being a part of a functional unit such as a gene or an open reading frame. Accordingly, by operably linking a promoter to a nucleic acid sequence encoding a polypeptide the two elements becomes part of the functional unit—a gene. The linking of the promoter to the nucleic acid sequence enables the transcription of the nucleic acid sequence directed by the promoter. By operably linking two heterologous nucleic acid sequences encoding a polypeptide the sequences becomes part of the functional unit—an open reading frame encoding a fusion protein comprising the amino acid sequences encoding by the by the heterologous nucleic acid sequences. By operably linking two amino acids sequences, the sequences become part of the same functional unit a polypeptide. Operably linking two heterologous amino acid sequences generates a hybrid (fusion) polypeptide.

### Reference

In order to determine the clinical severity of the abnormal cellular function, means for evaluating the detectable levels or amounts of Wrap53 measured can involve the use of a reference or reference means or base level. The reference can also facilitate measurements made in an assay and method variations, kit variations, handling variations and other variations not related directly or indirectly to the concentration of Wrap53.

In some contexts, the term “reference” relates to a standard in relation to quantity, quality or type, against which other values or characteristics can be compared, such as e.g. a standard curve.

A set of reference data is established by collecting the reference values for a number of samples. Non-limiting examples of types of references are 1) average values from a number of cancer cases with known prognosis or 2) average values from a number of normal cells. As will be obvious to those skilled in the art, the set of reference data will improve by including increasing numbers of reference values.

The skilled person will be able to set the reference level in a way such that a sample considered to be “equal to” the reference level, falls within the relevant group. Thus, it is to be understood that the in present invention the reference level may be adjusted in a way that samples falling within the group “equal to a reference level” can be positioned within the positive group and/or the negative group depending on the type of reference sample.

### Wrap53

Wrap53 is a 548 amino acid protein (NCBI Reference Sequence: NP 060551.2). Alternative names are WD repeat-containing protein 79, WD40 repeats-containing protein encoding RNA antisense to p53, WDR79 and TCAB 1. As used herein the term Wrap53 will be used. The protein sequence for Wrap53 (NCBI Reference Sequence: NP 060551.2) is disclosed in SEQ ID NO. 1 and the cDNA sequence for Wrap53 (NCBI Reference Sequence: NM 018081.2) is disclosed in SEQ ID NO. 2. It is to be understood that splice variants and fragments of Wrap53 may also be measured using the embodiments described herein. Similar, minor variations in the Wrap53 sequences may also be detected using the methods described herein. Minor variations could e.g. be SNP's. Also posttranslational modifcations such as phosporylations and methylations in the Wrap53 protein may be measured using the methods of the invention.

### Biological Sample

In the present context, the term “biological sample” relates to any liquid, liquefied or solid sample collected from an individual to be analyzed. Preferably the sample is a breast tissue sample and more preferably the sample is a breast cancer tissue sample. The tissue may be obtained from the open surgery, or prior to surgery from a fine needle biopsy (cells) or a core needle biopsy (tissue). Other sample types may be blood or urine. In one embodiment the sample is a sample in which blood Circulating Tumor Cells (CTC) may be isolated,

### Immunoassays

In their most simple and direct sense, immunoassays are binding assays. Antibody-binding to Wrap53 can be detected by any immunoassay means known in the art. Preferably, antibody binding is detected by an assay selected from the group consisting of protein microarray assay, radioimmunoassay (MA), enzyme-linked immunosorbent assay (ELISA), fluoroimmunoassay, immunofluorometric assay, immunohistochemical assays and immunoradiometric assay.

### Sequence Identity

The term “sequence identity” indicates a quantitative measure of the degree of homology between two amino acid sequences or between two nucleic acid sequences of equal length. If the two sequences to be compared are not of equal length, they must be aligned to give the best possible fit, allowing the insertion of gaps or, alternatively, truncation at the ends of the polypeptide sequences or nucleotide sequences. The sequence identity can be calculated as

$$\frac{( {N}_{ref} - {N}_{dif} ) 100}{{N}_{ref}} ,$$

wherein Ndif is the total number of non-identical residues in the two sequences when aligned and wherein Nref is the number of residues in one of the sequences. Hence, the DNA sequence AGTCAGTC (SEQ ID NO. 3) will have a sequence identity of 75% with the sequence AATCAATC (SEQ ID NO. 4) (Ndif=2 and Nref=8). A gap is counted as non-identity of the specific residue(s), i.e. the DNA sequence AGTGTC (SEQ ID NO. 5) will have a sequence identity of 75% with the DNA sequence AGTCAGTC (SEQ ID NO. 3) (Ndif=2 and Nref=8).

With respect all embodiments of the invention relating to nucleotide sequences, the percentage of sequence identity between one or more sequences may also be based on alignments using the clustalW software with default settings. For nucleotide sequence alignments these settings are: Alignment=3Dfull, Gap Open 10.00, Gap Ext. 0.20, Gap separation Dist. 4, DNA weight matrix: identity (IUB).

Alternatively, and as illustrated in the examples, nucleotide sequences may be analysed using programme DNASIS Max and the comparison of the sequences may be done at accessible sites on the internet. This service is based on the two comparison algorithms called Smith-Waterman (SW) and ParAlign. The first algorithm was published by Smith and Waterman (1981) and is a well established method that finds the optimal local alignment of two sequences. The other algorithm, ParAlign, is a heuristic method for sequence alignment. Default settings for score matrix and Gap penalties as well as E-values were used.

Aspects of the present invention relate to methods where a breast cancer sample is analysed for Wrap53 levels. Preferably the analysis is performed using immunohistochemical assay. The sample is analysed for levels of Wrap53 in the nucleus and in the cytoplasm. An indication of better survival is present when 1) an up-regulation or presence of Wrap53 in the nucleus is present, 2) a down-regulation or absence of Wrap53 in the cytoplasm is detected or 3) where an increased ratio of nuclear Wrap53 levels to cytoplasmic Wrap53 is detected. It is of course to be understood that the levels have to be compared to reference levels.

### Method for Determining the Prognosis of Breast Cancer

It has been discovered that Wrap53 is a prognostic marker for breast cancer survival by monitoring the levels or amounts of Wrap53 in the nucleus and/or cytoplasm of a cell (e.g., a breast tissue cell). Similarly, the ratio of Wrap53 levels in the nucleus to the Wrap53 levels in the cytoplasm may be used as a biomarker in breast cancer prognosis. Thus, some embodiments relate to a method for determining the prognosis of breast cancer comprising:
measuring the level of Wrap53 in a biological sample obtained from an individual,
comparing said level with a reference level, and
determining that the individual is likely to have a poor prognosis when:
1) the level of Wrap53 in the nucleus of cells in the sample is below the reference level,
2) the ratio between the level of Wrap53 in the nucleus of cells in the sample to the level of Wrap53 in the cytoplasm of cells in the sample is below the reference level, and/or
3) the level of Wrap53 in the cytoplasm of cells in the sample is above the reference level; or
determining that the individual is likely to have a good prognosis of breast cancer when:
1) the level of Wrap53 in the nucleus of cells in the sample is above or equal to the reference level,
2) the ratio between the level of Wrap53 in the nucleus of cells in the sample and the level of Wrap53 in the cytoplasm of cells in the sample is above or equal to the reference level, and/or
3) the level of Wrap53 in the cytoplasm of cells in the sample is below or equal to the reference level.

Thus, the levels of Wrap53 in the nucleus and/or the cytoplasm can be used to determine the prognosis of breast cancer. In a preferred embodiment Wrap53 levels is measured by immunoassays selected from the group consisting of protein microarray assay, radioimmunoassay (MA), enzyme-linked immunosorbent assay (ELISA), fluoroimmunoassay, immunofluorometric assay, immunohistochemical assays and immunoradiometric assay. The pathologist is likely to prefer immunohistochemical assays since antibody stainings can be combined with physical parameters in a sample such as cell size, tissue and cell morphology, and overall constitution of the sample. Again, it is to be understood that the methods described herein may be used for choosing a therapy for the disease, as an indication of whether to initiate a therapy, risk assessment of the disease, diagnostics, prognostics and predictive purposes.

Wrap53 levels may also be determined by measuring the RNA level of Wrap53 in the nucleus, the cytoplasm or both. Thus, in an embodiment of the invention, level of Wrap53 is determined by measuring the RNA levels of Wrap53 in the nucleus, the cytoplasm or both. Typical methods, known to the person skilled in the art, for measuring RNA levels are PCR, in situ hybridization, in situ PCR, QuantiGene® and RNA microarrays/chips.

### Wrap53 and ROC-Curves

In some embodiments, ROC-curves may be used to establish specificity and sensitivity of an assay. Thus, another aspect the invention relates to a method for determining the prognosis of breast cancer comprising:
a) measuring the level of Wrap53 in a biological sample obtained from an individual,
b) comparing said level with a reference level,
c) generating a ROC curve containing values representing the level of Wrap53 determined in the biological sample and the reference level,
d) selecting a desired sensitivity,
e) determining the specificity corresponding to the desired sensitivity from the ROC curve,
   determining that the individual is likely to have a poor prognosis when:
   1) the level of Wrap53 in the nucleus of cells in the sample is below the reference level corresponding to the desired specificity,
   2) the ratio between the Wrap53 in the nucleus of cells in the sample and the level of Wrap53 in the cytoplasm of cells in the sample is below the reference level corresponding to the desired specificity, and/or
   3) the level of Wrap53 in the cytoplasm of cells in the sample is above the reference level corresponding to the desired specificity; or
determining that the individual as likely to have a good prognosis of breast cancer when:
   1) the level of Wrap53 in the nucleus of cells in the sample is above or equal to the reference level corresponding to the desired specificity,
   2) the ratio between the Wrap53 in the nucleus of cells in the sample and the level of Wrap53 in the cytoplasm of cells in the sample is above or equal to the reference level corresponding to the desired specificity, and/or
   3) the level of Wrap53 in the cytoplasm of cells in the sample is below or equal to the reference level corresponding to the desired specificity.

By using ROC-curves the skilled person is able to establish specificity and sensitivity of the assay to a level he finds suitable in the present case.

In the above mentioned aspects, it is understood that the method may be used for choosing a therapy for the disease, as an indication of whether to initiate a therapy or as an indication to delay the initiation of a therapy.

### Determining the Predilection of a Breast Cancer

Similarly, some embodiments concern a method for determining the predilection for survival from breast cancer. Thus, some embodiments relate to a method for determining the predilection for survival of an individual with breast cancer comprising:
measuring, at a first time, the level of Wrap53 in a biological sample obtained from an individual that has breast cancer,
measuring, at a second time, the level of Wrap53 in a biological sample obtained from said individual,
comparing the level of Wrap53 measured at said first time to the level of Wrap53 measured at said second time, and
determining the predilection for survival of said individual, wherein:
   1) an increase in the level of Wrap53 in the nucleus of cells in the sample is indicative of an improved survival rate,
   2) a decrease in the level of Wrap53 in the nucleus of cells in the sample is indicative of a reduced survival rate,
   3) an unchanged level of Wrap53 in the nucleus of cells in the sample is indicative of an unchanged survival rate,
   4) an increase in the ratio between the level of Wrap53 in the nucleus of cells in the sample and the level of Wrap53 in the cytoplasm of cells in the sample is indicative of an improved survival rate,
   5) a decrease in the ratio between the level of Wrap53 in the nucleus of cells in the sample and the level of Wrap53 in the cytoplasm of cells in the sample is indicative of a reduced survival rate,
   6) an unchanged ratio between the level of Wrap53 in the nucleus of cells in the sample and the level of Wrap53 in the cytoplasm of cells in the sample is indicative of an unchanged survival rate,
   7) an increase in the level of Wrap53 in the cytoplasm of cells in the sample is indicative of a reduced survival rate,
   8) a decrease in the level of Wrap53 in the cytoplasm of cells in the sample is indicative of an improved survival rate, or
   9) an unchanged level of Wrap53 in the cytoplasm of cells in the sample is indicative of an unchanged survival rate.

By obtaining data at more than one time point the development of a breast cancer can be determined with respect to Wrap53. The development in the present aspect of the invention relates to whether there is an increase, a decline or no change of Wrap53 levels over time. The level of Wrap53 can be determined both in the nucleus, the cytoplasm and as a ratio of the level of WRAp53 in the nucleus to the level of Wrap53 in the cytoplasm. Samples may also be obtained more than two times such as three, four, five, six and seven times or more than seven times. The comparison can then be made between any of the obtained samples. Again, it is of course to be understood that the method may be used for choosing a therapy for the disease, as an indication of whether to initiate a therapy or as an indication to delay the initiation of a therapy.

### Treatment Between Measurements

It may be desired to provide a treatment regime between two measurements. Thus, in an embodiment one or more treatment regimes are provided to said individual between said first time and said second time Wrap53 levels are measured. By providing a treatment regime between two or more measurements the effect of the treatment can be determined. In this way the clinician may choose to change a current therapy, change the concentrations of the used drugs, or maintain the current treatment. Thus, some embodiments relate to the use of any of the methods described herein to determine an appropriate treatment for a particular patient having breast cancer. Many different treatments of breast cancer exist. Since Wrap53 levels are indicative of breast cancer prognosis, the clinician may choose a less aggressive treatment if the patient has a good prognosis (e.g., when significant or threshold quantities of WRAP53 are detected in the nucleus of breast tissue cells and/or when the cytoplasm is significantly depleted of WRAP53), whereas more aggressive treatments may be necessary if the patient has a poor prognosis (e.g., when significant or threshold quantities of WRAP53 are detected in the cytoplasm of breast tissue cells and/or when the nucleus is significantly depleted of WRAP53). Threshold or significant levels of WRAP53 can be determined statistically by reviewing a database containing WRAP53 measurements of a plurality of breast cancer patients and/or by referring to a reference or baseline. Again, it is of course to be understood that the methods described herein may be used for choosing a therapy for the disease, as an indication of whether to initiate a therapy, risk assessment of the disease, diagnostics, prognostics and predictive purposes.

### Sample Type

In the present context, the term “sample” relates to any liquid or solid sample collected from an individual to be analyzed. Preferably, the sample is liquefied at the time of assaying.

In another embodiment of the present invention, a minimum of handling steps of the sample is necessary before measuring the level of Wrap53. In the present context, the subject “handling steps” relates to any kind of pre-treatment of the liquid sample before or after it has been applied to the assay, kit or method. Pre-treatment procedures includes separation, filtration, dilution, distillation, concentration, inactivation of interfering compounds, centrifugation, heating, fixation, addition of reagents, or chemical treatment.

In accordance with the present invention, the sample to be analyzed is collected from any kind of mammal, including a human being, a pet animal, a zoo animal and a farm animal.

In yet another embodiment of the present invention, the sample is derived from any source such as body fluids.

Preferably, this source is selected from the group consisting of milk, semen, blood, serum, plasma, saliva, urine, sweat, ocular lens fluid, cerebral spinal fluid, cerebrospinal fluid, ascites fluid, mucous fluid, synovial fluid, peritoneal fluid, vaginal discharge, vaginal secretion, cervical discharge, cervical or vaginal swab material or pleural, amniotic fluid and other secreted fluids, substances and tissue biopsies from organs such as the brain, heart and intestine.

In cases where the breast cancer has metastasized it may be of clinical value to determine the origin of such cancer for treatment purposes, thus it may be necessary to take samples from literally any organ/tissue these metastasized cells could be present in.

In one embodiment of the present invention relates to a method according to the present invention, wherein said body sample or biological sample is selected from the group consisting of blood, urine, pleural fluid, oral washings, vaginal washings, cervical washings, tissue biopsies, and follicular fluid.

Another embodiment of the present invention relates to a method according to the present invention, wherein said biological sample is selected from the group consisting of urine, saliva, blood, plasma and serum.

The sample taken may be dried for transport and future analysis. Thus the method of the present invention includes the analysis of both liquid and dried samples.

The type of sample used for using Wrap53 as a biomarker in breast cancer of course influence the assay. Thus, in an embodiment the biological sample is a breast tissue sample.

In a preferred embodiment, the sample is a breast tumor tissue sample.

The tissue may be obtained from the open surgery, or prior to surgery from a fine needle biopsy (cells) or a core needle biopsy (tissue).

Healthy tissue, blood or urine may be used. Potential analysis of fluids may involve measuring expression of the WRAP53 protein and/or RNA (or associated profiles), or in the nearby future analysing Circulating Tumor Cells (CTC) isolated from the blood.

The person skilled in art knows how to obtain, preserve and store such samples in a way allowing the measurements to be obtained at later stages. Examples of typical tissue preservative methods include formalin fixation followed by paraffin embedding (FFPE), freezing (preferably in the presence of cryopreservatives), ethanol fixation, acetone fixation and methanol fixation.

Similarly, the skilled person will know of methods to unmask potential epitopes in a sample which have been preserved by standard methods. Examples of unmasking epitopes and/or reverting crosslinkage include heating and microwaves.

Wrap53 Combined with Other Biomarkers

The prognostic potential of the present invention may be strengthen by combining the Wrap53 measurements with other biomarkers related to cancer or more specifically related to breast cancer. Thus, in an embodiment the methods further comprise measuring at least one biomarker selected from the group consisting of the biomarkers TP53, Ki67, ER (Estrogen receptor), PR (Progesterone receptor), HER2 (human epidermal growth factor receptor 2), the clinical markers age, tumor size, lymph node status, the histological markers tumor type or tumor grade and TP53 mutational status in a biological sample obtained from said individual. HER2 (human epidermal growth factor receptor 2) is also known as ErbB-2, ERBB2 and Her2/neu.

In the present context, the term “biomarker” relates both to clinical markers such as age, tumor size and lymph node status, histological markers such as tumor type and typical biomarkers such as TP53 mutational status, ER, PR and HER2/ERBB2/Neu.

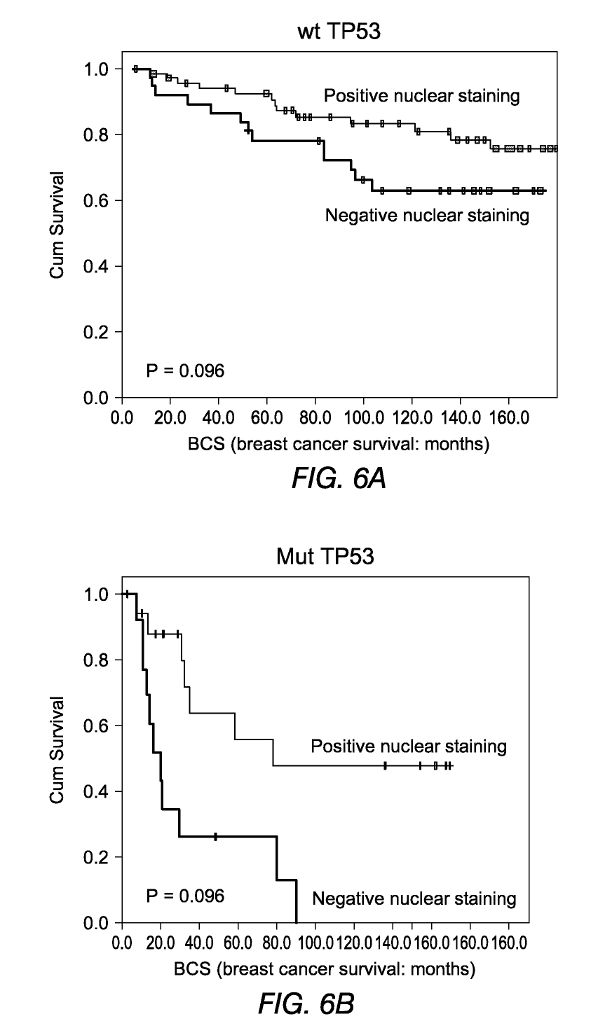

Patients with a TP53 mutation in the tumor have poor prognosis (vs. TP53 wt). Wrap53 nuclear staining may be an important prognostic marker in patients with a TP53 mutation in the tumor (FIG. 6). Treatment may be affected by such knowledge, avoiding over-/under-treatment and give more personalized therapy.

Thus, in an additional embodiment, Wrap53 levels are combined with TP53 mutational status.

Two genetic tests for breast cancer, MammaPrint® and Oncotype DX® are increasingly popular especially in US and are based on a profile/set of biomarkers. The skilled person knows how to incorporate other biomarkers that would be relevant to combine with Wrap53 levels.

### Antibodies

New epitopes on Wrap53 that are highly robust sites for the detection of Wrap53 have been discovered. Accordingly, some embodiments relate to an isolated polynucleotide comprising a nucleic acid sequence encoding a polypeptide capable of specifically binding to an exposed epitope on Wrap53, wherein said epitope consists of, consists essentially of, or comprises at least part of position 483-496 or 449-463 of SEQ ID NO.1. These sequence elements correspond to the epitopes toward which the antibodies of example 1 and 2 were raised, indicating that these sequence elements are good epitopes on Wrap53 for breast cancer prognosis. Binding fragments of these antibodies are also embodiments (e.g., Fab fragment, an Fv fragment, a single chain antibody, a single domain antibody).

Some embodiments relate to an isolated polynucleotide comprising a nucleic acid sequence encoding a polypeptide capable of specifically binding an exposed epitope on Wrap53, wherein said epitope has at least 50% sequence identity with position 483-496 or 449-463 of SEQ ID NO.1 or such as at least 60%, such as at least 70%, such as at least 80%, such as at least 90% such as at least 95% or such as 100% sequence identity with position 483-496 or 449-463 of SEQ ID NO.1.

Similarly, an aspect of the present invention relates to an isolated polypeptide capable of specifically binding an exposed epitope on Wrap53, wherein said epitope consists of, consists essentially of, or comprises at least part of position 483-496 or 449-463 of SEQ ID NO.1.

These sequence elements corresponds to the epitopes toward which the antibodies of example 1 and 2 were raised, indicating that these sequence elements are good epitopes on Wrap53 for breast cancer prognosis.

Some embodiments relate to an isolated polypeptide capable of specifically binding an exposed epitope on Wrap53, wherein said epitope has about at least 50% sequence identity with position 483-496 or 449-463 of SEQ ID NO.1 or such as about at least 60%, such as about at least 70%, such as about at least 80%, such as about at least 90% such as about at least 95% or such as 100% sequence identity with position 483-496 or 449-463 of SEQ ID NO.1.

The polypeptides described herein may be operably or indirectly linked to one or more reporter moieties which will be well known to those skilled in the art, as will methods by which they may be attached to the antibodies of the invention. A suitable reporter moiety may be selected from the group consisting of a fluorescent moiety; a luminescent moiety; a bioluminescent moiety; a radioactive material; a prosthetic group; a colorimetric moiety; a nanoparticles having suitable detectable properties, and a chromogenic moiety.

One suitable manner by which antibodies of the invention may be indirectly labelled is by means of a “primary antibody ‘V’ secondary” antibody strategy. Briefly, in such a strategy the unlabelled polypeptide of the invention is used as a “primary antibody” able to bind to Wrap53 in a sample (e.g. a patient sample). A labelled “secondary antibody” (chosen to react solely with the antibody of the invention, and not with other materials in the sample) is then used to bind to the primary antibody. Thus, an unlabelled antibody described herein is effectively bound to the label attached to the secondary antibody.

The desired polypeptide may be expressed by a nucleic acid construct in vitro or in vivo. Thus, some embodiments concern a nucleic acid construct comprising the isolated polynucleotide according to the invention, and a promoter for directing an expression of said isolated polynucleotide in cells.

The polypeptide described herein may have several origins. Thus, some embodiments relate to a polypeptide, wherein said polypeptide is selected from the group consisting of a Fab fragment, an Fv fragment, a single chain antibody, a single domain antibody and an antibody.

The antibodies according to the invention may be both polyclonal and monoclonal.

### Diagnostic Kit

A kit comprising an antibody against the specific epitopes discovered by the inventors may be relevant for e.g. prognostic purposes. Thus in yet an aspect of the invention relates to a kit for indicating a breast cancer prognosis or determining the course of a breast cancer comprising:
an antibody against at least part of position 483-496 or 449-463 of Wrap53 (SEQ ID NO.1),
an instruction manual explaining how to carry out at least one of the methods according to the preceding claims.

A diagnostic kit as described above can be important for improving diagnosis/prognosis for breast cancer patients. Again, it is to be understood that the diagnostic kit may be used for choosing a therapy for the disease, as an indication of whether to initiate a therapy, risk assessment of the disease, diagnostics, prognostics and predictive purposes.

In an embodiment the invention relates to an diagnostic kit comprising an antibody capable of specifically binding an exposed epitope on Wrap53, wherein said epitope has about at least 50% sequence identity with position 483-496 or 449-463 of SEQ ID NO.1 or such as about at least 60%, such as about at least 70%, such as about at least 80%, such as about at least 90% such as about at least 95% or such as 100% sequence identity with position 483-496 or 449-463 of SEQ ID NO.1.

### Method of Determining the Predilection for Survival for an Individual Having Breast Cancer

Wrap53 may be used as a general marker for breast cancer prognosis. Unexpectedly, the inventors have discovered that the relationship between the levels of Wrap53 in the nucleus to the levels of wrap53 in the cytoplasm is important for cancer prognosis. Thus, some embodiments relate to a method of determining the predilection for survival for an individual having breast cancer comprising:
obtaining a breast tissue sample from said individual,
   measuring the amount of Wrap53 in the nucleus of cells in said breast tissue sample,
   measuring the amount of Wrap53 in the cytoplasm of cells in said breast tissue sample,
   comparing the amount Wrap53 measured in the nucleus of cells in said breast tissue sample to the amount of Wrap53 measured in the cytoplasm of cells in said breast tissue sample, and
   determining the predilection for survival for said individual having breast cancer.

### Method of Evaluating a Treatmen Protocol for an Individual Having Breast Cancer

Wrap53 may also be used for evaluating a treatment protocol for breast cancer patients. Thus, some embodiments relate to a method of evaluating a treatment protocol for an individual having breast cancer comprising:
obtaining a first breast tissue sample from an individual having breast cancer prior to initiating a treatment protocol for breast cancer,
measuring the amount of Wrap53 in the nucleus of cells in said first breast tissue sample,
measuring the amount of Wrap53 in the cytoplasm of cells in said first breast tissue sample,
comparing the amount Wrap53 measured in the nucleus of cells in said first breast tissue sample to the amount of Wrap53 measured in the cytoplasm of cells in said first breast tissue sample,
obtaining a second breast tissue sample from said individual after initiation or completion of said treatment protocol for breast cancer,
measuring the amount of Wrap53 in the nucleus of cells in said second breast tissue sample,
measuring the amount of Wrap53 in the cytoplasm of cells in said second breast tissue sample,
comparing the amount Wrap53 measured in the nucleus of cells in said second breast tissue sample to the amount of Wrap53 measured in the cytoplasm of cells in said second breast tissue sample, and
determining the progress of said treatment protocol by comparing the amounts of Wrap53 measured in the nucleus and/or cytoplasm of cells in said second breast tissue sample to the amount of Wrap53 measured in the nucleus and/or cytoplasm of cells in said first breast tissue sample.

Thus, by providing a treatment regime between two or more measurements the effect of the treatment can be determined. In this way the clinician may choose to change a current therapy, change the concentrations of the used drugs, or maintain the current treatment.

### Ratio of the Amount of Wrap53 Measured in the Nucleus of Cells to the Amount of Wrap53 Measured in the Cytoplasm

As mentioned earlier, the ratio between the amounts of Wrap53 in the nucleus to the amount in the cytoplasm is important when making a breast cancer prognosis. Thus, some embodiments relate to a method, wherein the ratio of the amount of Wrap53 measured in the nucleus of cells in said breast tissue sample to the amount of Wrap53 measured in the cytoplasm of cells in said breast tissue sample is determined.

### References

As also described earlier, it may be necessary to compare the measured Wrap53 levels to one or more reference levels. Thus, in an embodiment the methods further comprising comparing the amount of Wrap53 measured in the nucleus and/or cytoplasm of the cells in said breast tissue samples or the ratio of the amount of Wrap53 measured in the nucleus of cells in said breast tissue sample to the amount of Wrap53 measured in the cytoplasm of cells in said breast tissue sample with a reference level.

As will be generally understood by those of skill in the art, methods for screening for breast cancer prognosis are processes of decision making by comparison. For any decision making process, reference values based on patients having the disease or condition of interest and/or patients not having the disease or condition of interest are needed.

In one preferred embodiment of the present invention, the reference means is an internal reference means and/or an external reference means.

In the present context the term “internal reference means” relates to a reference which is not handled by the user directly for each determination but which is incorporated into a device for the determination of the prognostic value of Wrap53, whereby only the ‘final result’ or the ‘final measurement’ is presented. The terms the “final result” or the “final measurement” relates to the result presented to the user when the reference value has been taken into account.

In a further embodiment of the present invention, the internal reference means is provided in connection to a device used for the determination of the prognostic value of Wrap53.

In yet an embodiment of the present invention the device is selected from the group consisting of an assay, a stick, a dry-stick, an electrical device, an electrode, a reader (spectrophotometric readers, IR-readers, isotopic readers and similar readers), histochemistry, and similar means incorporating a reference.

In the present context, the term “external reference means” relates to a reference which is handled directly by the user in order to determine the prognostic value of Wrap53, before obtaining the ‘final result’ or the ‘final measurement’

In yet a further embodiment of the present invention external reference means are selected from the group consisting of a table, a diagram and similar reference means where the user can compare the measured signal to the selected reference means. The external reference means relates to a reference used as a calibration, value reference, information object, etc. for Wrap53 and which has been excluded from the device used.

One embodiment of the present invention relates to a method according to the present invention, wherein said reference level/predetermined value is indicative of a normal physiological condition of said individual.

One embodiment of the present invention relates to a method according to the present invention, wherein said reference level/predetermined value is indicative of a condition is a breast cancer of said individual.

Although any of the known analytical methods for measuring the prognostic value of Wrap53 will function in the present invention, as apparent to one skilled in the art, the analytical method used for Wrap53 must be the same method used to generate the reference data for Wrap53. If a new analytical method is used for Wrap53, a new set of reference data, based on data developed with the method, must be generated. Thus, the technique utilized to analyze the blood should be the same for the reference data and the samples to be screened

### Risk Assessment

The present inventors have successfully developed a new ELISA method to measure the prognostic value of Wrap53 in breast cancer. To determine whether the individual is at increased risk of poor prognosis, a cut-off must be established. This cut-off may be established by the laboratory, the physician or on a case by case basis by each patient.

The cut-off level can be based on several criteria including the number of women who would go on for further invasive diagnostic testing, the average risk of carrying a breast tumor to all the women who go on for further invasive diagnostic testing, a decision that any woman whose patient specific risk is greater than a certain risk level such as e.g. about 1 in 400 or about 1:250 (as defined by the screening organisation or the individual woman) should go on for further invasive diagnostic testing or other criteria known to those skilled in the art.

The cut-off level could be established using a number of methods, including: percentiles, mean plus or minus standard deviation(s); multiples of median value; patient specific risk or other methods known to those who are skilled in the art.

The multivariate discriminant analysis and other risk assessments can be performed on the commercially available computer program statistical package Statistical Analysis system (manufactured and sold by SAS Institute Inc.) or by other methods of multivariate statistical analysis or other statistical software packages or screening software known to those skilled in the art.

As obvious to one skilled in the art, in any of the embodiments discussed above, changing the risk cut-off level of a positive or using different a priori risks which may apply to different subgroups in the population, could change the results of the discriminant analysis for each patient.

The stability tests described herein demonstrate that ADAM12 is highly stable with routine handling; thus, the present inventors conclude that ADAM12 is an attractive analyte for clinical use. The data presented here suggest that ADAM12 is a potentially valuable marker for use in prenatal screening.

### ROC Curves

ROC curves a standard way of establishing specificity and sensitivity in biomarker assays. Thus, in an embodiment the methods further comprise generating a ROC curve containing values representing the amounts of Wrap53 measured in the nucleus and/or cytoplasm of the cells in said breast tissue samples.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

The Wrap53 protein is detected using Wrap53 (483) and Wrap53 (4491 antibodies

### Material and Methods:

To generate the Wrap53 (483) antibody rabbits were immunized with a KLH-conjugated Wrap53 peptide (NH2-) (C) RVFPEPTESGDEGE (SEQ ID NO. 6) (—CONH2), corresponding to amino acids 483-496 of full-length Wrap53 protein, followed by affinity IgG purification (Innovagen AB, Sweden). A Cysteine (C) residue was added in the N terminus to facilitate coupling of the peptide to the carrier molecule.

To generate the Wrap53 (449) antibody rabbits were immunized with a KLH-conjugated Wrap53 peptide (Ac-) GKPEPVLSFLPQKDC (SEQ ID NO. 7) (—COOH), corresponding to amino acids 449-463 of full-length Wrap53 protein, followed by affinity IgG purification (Innovagen AB, Sweden).

### Conclusion:

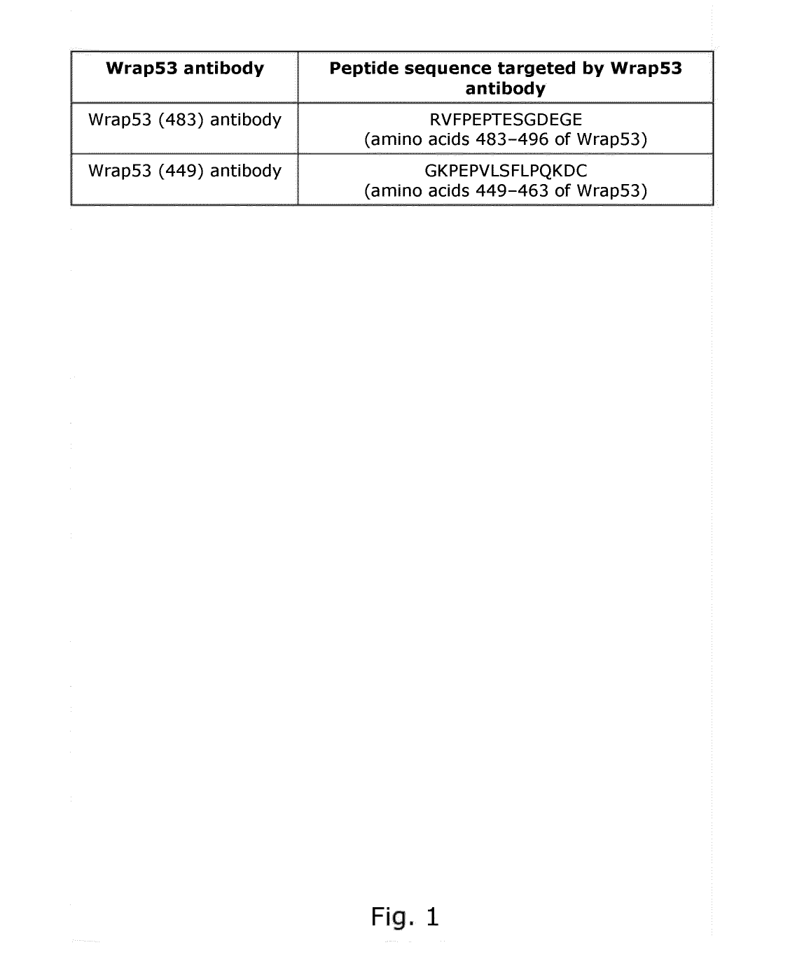

The above peptide sequences (FIG. 1) are unique for the Wrap53 protein and therefore the Wrap53 (483) and (449) antibodies should specifically recognize the Wrap53 protein.

### Example 2

The Wrap53 (483) and Wrap53 (449) antibodies specifically detects the Wrap53 protein.

### Material and Methods:

Cell extracts for Western blot analysis were prepared by harvesting cells, wash once in PBS and lyse the cells in ice cold WB lysis buffer (100mM Tris-HCL pH 8, 150mM NaC1, 1% NP-40, 1% PMSF, 1% protease inhibitor cocktail) for 30 minutes on ice. Lysates were centrifuged at 14000rpm for 15 minutes at 4° C. and protein concentrations were determined using Bradford assay (Biorad). Western blot analysis was performed according to standard procedures. Wrap53 knockdown was performed by transfected cells with 10-20nM of Wrap53-specific siRNA oligos using Oligofectamine (Invitrogen®) transfection reagent in accordance with the supplier's recommendations. The two siRNA oligonucleotides siWrap53#1 (targets Wrap53 sequence 5′- CCGGGAGAACCCGATTCATAT -3′, (SEQ ID NO. 8) cat# SI00388941) and siWrap53#2 (targets Wrap53 sequence 5′-AACGGGAGCCTTTCTGAAGAA -3′, (SEQ ID NO. 9) cat# SI00388948) were ordered from Qiagen®. Wrap53 overexpression was performed by transfecting expression 1 ug CMV plasmid containing Wrap53 open reading frame into cells using Lipofectamine 2000 Reagent (Invitrogen®) following the manufacturer's instructions. Generation of the CMV-Wrap53 plasmid has previously been described (Mahmoudi et al., 2009).

### Conclusion:

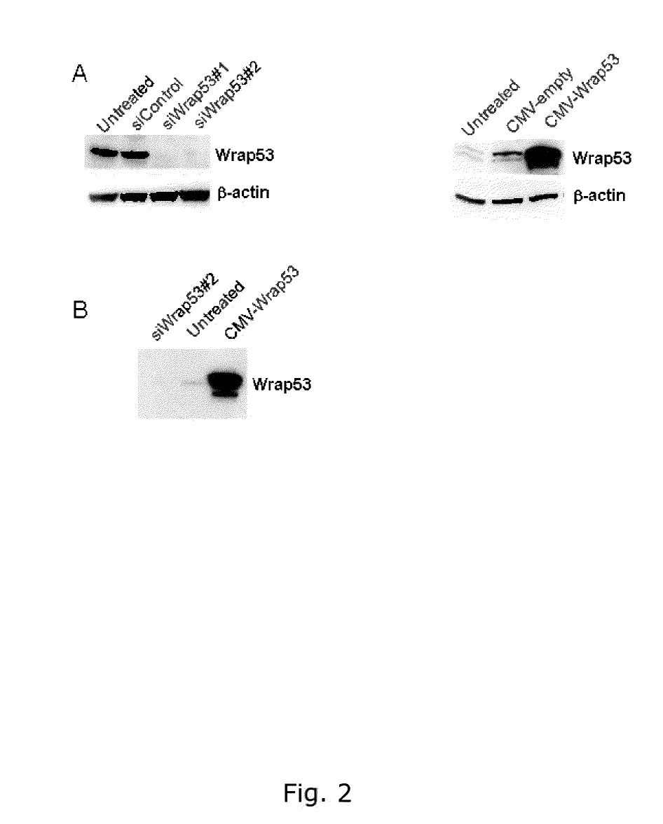

The Wrap53 (483) and (449) antibodies specifically recognize the Wrap53 protein, as demonstrated by loss of signal when knocking down the Wrap53 protein and gain of signal when overexpressing the Wrap53 protein (FIG. 2).

### Example 3

Wrap53 protein is expressed in the nucleus and in the cytoplasm in breast cancer cells (MCF-7) in normal fibroblasts (HDF)

### Material and Methods:

For immunofluorescence (IF) experiments, cells were grown on sterilized cover slips and fixed with 100% MeOH for 20 minutes at −20° C. The cells were then permeabilized with 0.1% Triton X-100 for 5 minutes at RT, followed by 30 minutes of blocking in blocking buffer (2% BSA, 5% glycerol, 0,2% Tween20, 0,1% NaN3). Cover slips were subsequently incubated for 1 hour in primary antibody and 40 minutes in secondary antibody diluted in blocking buffer. The cover slips were mounted with Vectorshield mounting medium with DAPI (Vector laboratories). Images were acquired with a Zeiss Axioplan 2 microscope, equipped with an AxioCam HRm Camera using 43 or 60 oil immersion lenses, and processed using Axiovision Release 4.7. A rabbit polyclonal Wrap53 (1-50) antibody from Bethyl Laboratories (#A301-442A) was used for the staining.

### Conclusions:

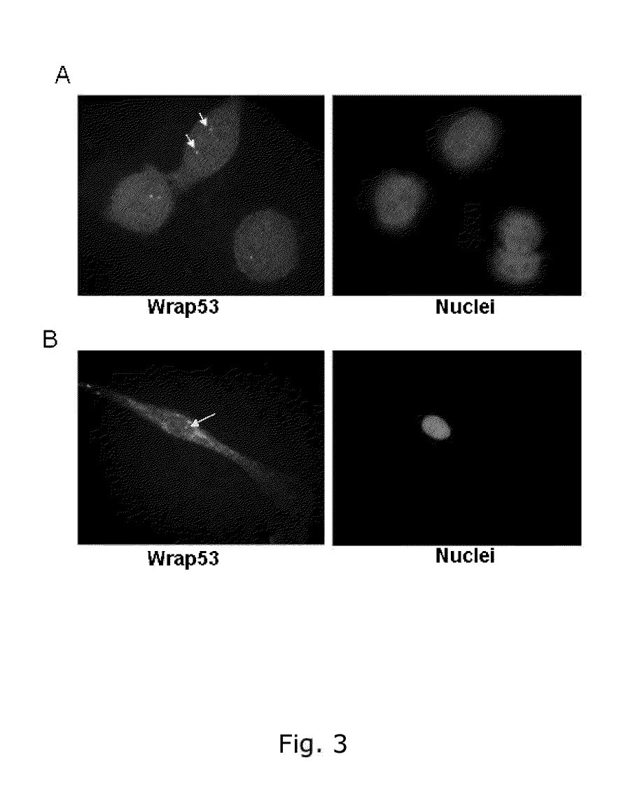

Wrap53 protein is diffusely expressed both in the nucleus and in the cytoplasm in breast cancer cells (MCF-7) and in normal fibroblasts (HDF). In the nucleus Wrap53 is enriched in nuclear structures called Cajal bodies (indicated with white arrows) (FIG. 3). A rabbit polyclonal Wrap53 (1-50) antibody from Bethyl Laboratories (#A301-442A) was used for the stainings.

### Example 4

The Wrap53 protein is expressed in both nucleus and cytoplasm of breast tumor cells.

### Material and Methods:

Tissue Microarrray (TMA) was constructed of three paraffin cores from each of totally 170 primary breast cancer patients and immunohisto-chemically (IHC) stained using polyclonal Wrap53 antibody (483). The IHC staining in nucleus and cytoplasm were scored separately using microscopy. The scoring were performed according to percentage of positively stained cells (0: 0% cells, 1: <5% cells, 2: 5-50% cells, 3: >50% cells). The minimum requirement to be scored was presence of more than 50 cancer cells in the core.

### Results:

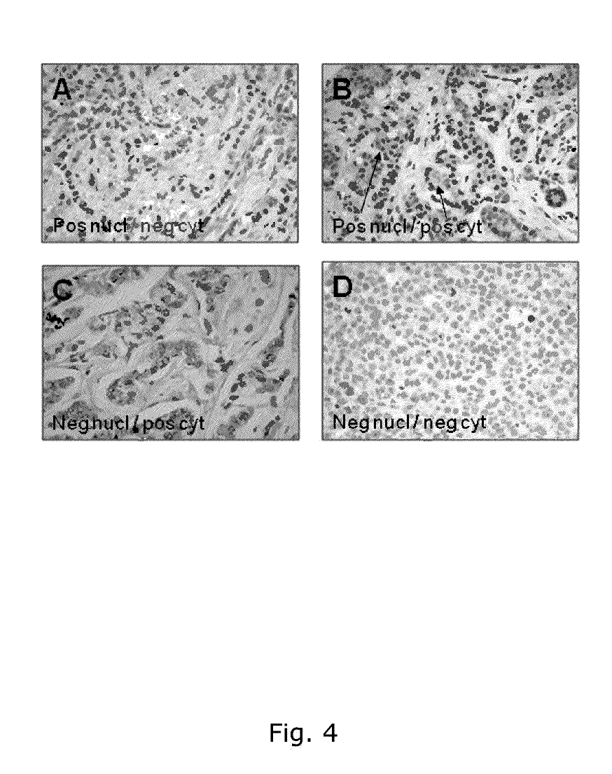

The Wrap53 protein was localized both in the nucleus and the cytoplasm of the breast carcinoma cells (FIG. 4). The series of 170 breast carcinomas showed a distribution of cases as follows (nucleus/cytoplasm): 39.6% pos/pos (A), 20.1% pos/neg (B), 20.8% neg/pos (C) and 19.5% neg/neg (D). A series of 860 breast cancer cases was used for validation.

### Conclusion:

A diverse pattern of immunohistochemistry staining was observed in the two series of breast primary breast tumor samples, representing various combinations of nuclear and/or cytoplasmic expression of the Wrap53 protein.

### Example 5

The Wrap53 protein is a marker for breast cancer survival, where Wrap53 in the nucleus predicts good survival. negative nucleus combined with positive cytoplasm predicts poor survival.

### Methods:

Positive nuclear scoring were defined as score 1-3 (0% threshold). Positive cytoplasmic staining were defined as score 2-3 (5% threshold), whereas negative cytoplasmic staining included the weak positive cases score 0-1. The different threshold is based on less distinct staining of cytoplasm compared to nucleus. Statistical analysis of results from IHC was performed using SPSS 15.0.

### Results:

Univariate survival analysis showed that breast cancer patients with positive nuclear staining of the Wrap53 protein had a statistically significant better survival than patients with negative nuclear staining (test series, p=0.003).

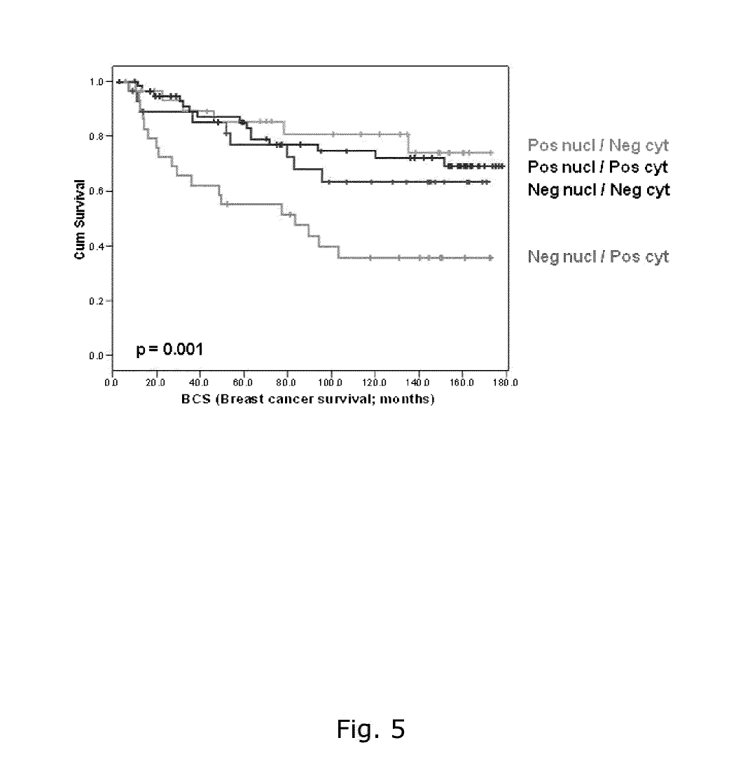

Combining results from staining of nucleus and cytoplasm showed that patients with tumors having positive nucleus/negative cytoplasm had the best prognosis, whereas those with negative nucleus/positive cytoplasm had the poorest prognosis (FIG. 5). Validation of the results in a series of 860 breast cancer, of which 690 where scoreable, supported the results (p=0.036).

### Conclusion:

Nuclear expression of the Wrap53 protein shows a significant impact on breast cancer survival. The effect on outcome seems to be related to the presence of Wrap53 in the nucleus rather than the lack of the protein in the cytoplasm, although this needs to be further explored. The stronger nuclear staining/higher score (0, 1, 2, 3), the better prognosis.

### Example 6

The Wrap53 nuclear expression is a significant prognostic marker in patients with TP53 mutant tumors.

### Methods:

Statistical analysis of results from IHC was performed using SPSS 15.0, producing Kaplan-Meier plot and obtaining p-value from the log rank test.

### Results:

Stratification by TP53 mutation status showed that Wrap53 nuclear staining is a strong prognostic marker in the TP53 mutant samples (p=0.005) (FIG. 6), but no statistically significant association was observed between nuclear Wrap53 expression and type of TP53 mutation.

### Conclusion:

Breast cancer patients with a TP53 mutation in their tumor have poor prognosis. Wrap53 nuclear staining may be an important prognostic marker in patients with a TP53 mutation in the tumor, and impact choice of treatment by differentiating between patients with good and poor outcome. Possibly over-/under-treatment could be reduced.

### Example 7

Wrap53 is a strong independent prognostic marker vs. all commonly applied clinicopathological and molecular markers of breast cancer.

### Methods:

Univariate and multivariate survival analyses were performed using the Cox regression model.

### Results:

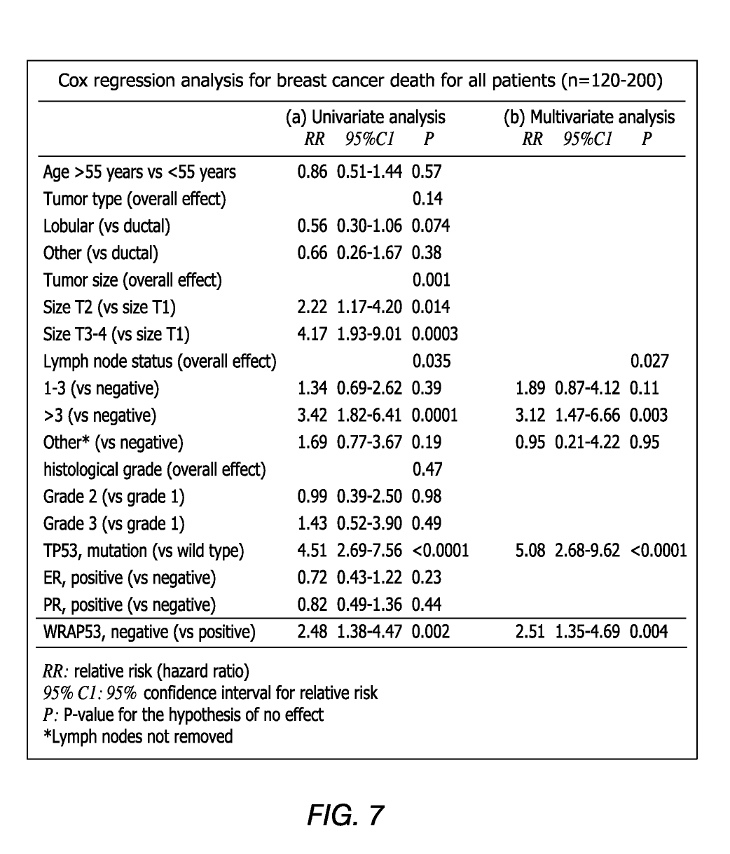

TP53 mutation status, Wrap53 nuclear protein and lymph node status were the significant remaining factors in a multivariate Cox regression model obtained after elimination of all non-significant factors (FIG. 7).

### Conclusion:

Wrap53 is a strong prognostic marker of breast cancer and may provide additional prognostic /predictive information supplementing currently established clinical markers.

### Example 8

The WRAP53 antibody (483) gives better immunohistochemical staining than the commercially available antibodies tested, showing less unspecific staining.

### Material and Methods:

Tissue Microarrray (TMA) was constructed of three paraffin cores from each of totally 170 primary breast cancer patients and immunohistochemically (IHC) stained using polyclonal WRAP53 antibody (483) and commercially polyclonal WRAP53 antibody (1-50). Both antibodies were tested for unspecific binding using blocking peptide. The IHC staining in nucleus and cytoplasm were scored separately using microscopy.

### Results:

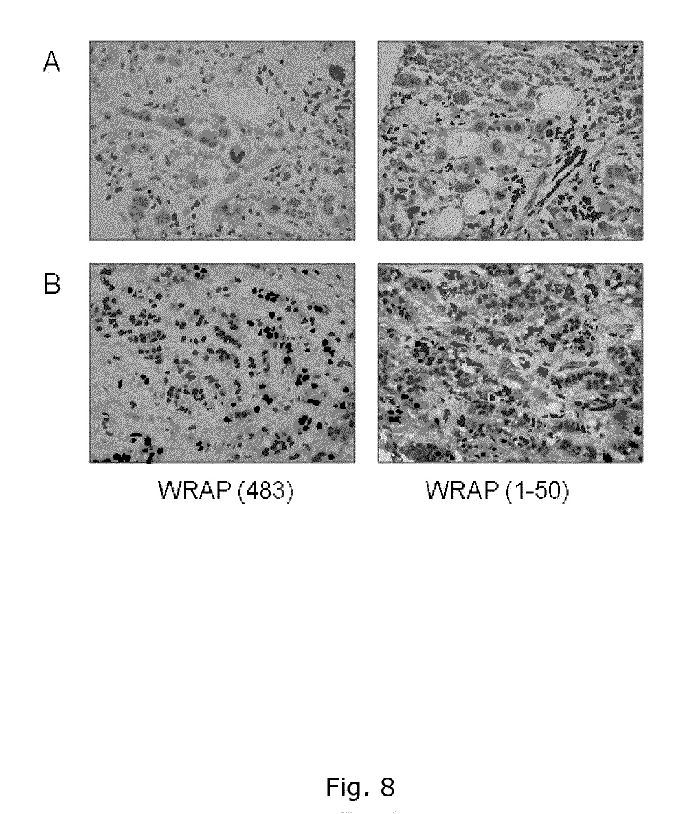

The IHC scoring was considered best by the pathologist using WRAP53 antibody (483) compared to the commercial WRAP53 antibody (1-50) from Bethyl Laboratories (#A301-442A). The WRAP53 antibody (483) was easier to score based on less unspecific binding and reduced background staining. The staining pattern was similar, but not as good (FIG. 8). There were almost no samples being neg nucl/neg cyto using the commercial antibody compared to WRAP53 antibody (483), illustrating the general impression of more diffuse unspecific staining.

### Conclusion:

A comparable diverse pattern of immunohistochemistry staining was observed in the 170 samples using the two different antibodies. The WRAP53 (483) antibody was generally considered to be best since it gave a more distinct pattern that improved the IHC scoring. Thus, the specific epitopes targeted by the antibodies of the invention appears to give better staining,

### REFERENCES

Zhang, Bioinformatics, (20) p 2390-8, 10 2004.
Montserrat Garcia-Closas et al. Common genetic variation in TP53 and its flanking genes, WDR79 and ATP1B2, and susceptibility to breast cancer and ATP1B2, and susceptibility to breast cancer. Int. J. Cancer: 121, 2532-2538 (2007).
WO 2008/041933 A2
Farnebo M Wrap53, a novel regulator of p53. Cell Cycle. 2009 Aug; 8(15):2343-6.
Mahmoudi, S., Henriksson, S., Corcoran, M, Mendez-Vidal, C., Wiman, K G., and Farnebo, M (2009). Wrap53, a natural p53 antisense transcript required for p53 induction upon DNA damage. Mol Cell 33, 462-471.

What is claimed is:

1. A method for determining the prognosis of breast cancer comprising:
a) measuring the level of Wrap53 in a biological sample obtained from an individual,
b) comparing said level with a reference level, and
c) determining that the individual is likely to have a poor prognosis when:
1) the level of Wrap53 in the nucleus of cells in the sample is below the reference level,
2) the ratio between the level of Wrap53 in the nucleus of cells in the sample to the level of Wrap53 in the cytoplasm of cells in the sample is below the reference level, or
3) the level of Wrap53 in the cytoplasm of cells in the sample is above to the reference level; or
d) determining that the individual is likely to have a good prognosis of breast cancer when:
1) the level of Wrap53 in the nucleus of cells in the sample is above or equal to the reference level,
2) the ratio between the level of Wrap53 in the nucleus of cells in the sample and the level of Wrap53 in the cytoplasm of cells in the sample is above or equal to the reference level, or
3) the level of Wrap53 in the cytoplasm of cells in the sample is below or equal to the reference level.

2. A method for determining the prognosis of breast cancer comprising:
a) measuring the level of Wrap53 in a biological sample obtained from an individual,
b) comparing said level with a reference level,
c) generating a ROC curve containing values representing the level of Wrap53 determined in the biological sample and the reference level,
d) selecting a desired sensitivity,
e) determining the specificity corresponding to the desired sensitivity from the ROC curve,
f) determining that the individual is likely to have a poor prognosis when:
1) the level of Wrap53 in the nucleus of cells in the sample is below the reference level corresponding to the desired specificity,
2) the ratio between the Wrap53 in the nucleus of cells in the sample and the level of Wrap53 in the cytoplasm of cells in the sample is below the reference level corresponding to the desired specificity, or
3) the level of Wrap53 in the cytoplasm of cells in the sample is above the reference level corresponding to the desired specificity; or
g) determining that the individual as likely to have a good prognosis of breast cancer when:
1) the level of Wrap53 in the nucleus of cells in the sample is above or equal to the reference level corresponding to the desired specificity,
2) the ratio between the Wrap53 in the nucleus of cells in the sample and the level of Wrap53 in the cytoplasm of cells in the sample is above or equal to the reference level corresponding to the desired specificity, or
3) the level of Wrap53 in the cytoplasm of cells in the sample is below or equal to the reference level corresponding to the desired specificity.

3. A method for determining the predilection for survival of an individual with breast cancer comprising:
a) measuring, at a first time, the level of Wrap53 in a biological sample obtained from an individual that has breast cancer,
b) measuring, at a second time, the level of Wrap53 in a biological sample obtained from said individual,
c) comparing the level of Wrap53 measured at said first time to the level of Wrap53 measured at said second time, and
d) determining the predilection for survival of said individual, wherein:
1) an increase in the level of Wrap53 in the nucleus of cells in the sample is indicative of an improved survival rate,
2) a decrease in the level of Wrap53 in the nucleus of cells in the sample is indicative of a reduced survival rate,
3) an unchanged level of Wrap53 in the nucleus of cells in the sample is indicative of an unchanged survival rate,
4) an increase in the ratio between the level of Wrap53 in the nucleus of cells in the sample and the level of Wrap53 in the cytoplasm of cells in the sample is indicative of an improved survival rate,
5) a decrease in the ratio between the level of Wrap53 in the nucleus of cells in the sample and the level of Wrap53 in the cytoplasm of cells in the sample is indicative of a reduced survival rate,
6) an unchanged ratio between the level of Wrap53 in the nucleus of cells in the sample and the level of Wrap53 in the cytoplasm of cells in the sample is indicative of an unchanged survival rate,
7) an increase in the level of Wrap53 in the cytoplasm of cells in the sample is indicative of a reduced survival rate,
8) a decrease in the level of Wrap53 in the cytoplasm of cells in the sample is indicative of an improved survival rate, or
9) an unchanged level of Wrap53 in the cytoplasm of cells in the sample is indicative of an unchanged survival rate.

4. The method according to claim 3, wherein one or more treatment regimes are provided to said individual between said first time and said second time Wrap53 levels are measured.

5. The method according claim 1, wherein said biological sample is a breast tissue sample.

6. The method of claim 1, further comprising measuring at least one biomarker selected from the group consisting of: TP53, Ki67, ER (Estrogen receptor), PR (Progesterone receptor), HER2 (human epidermal growth factor receptor 2), the clinical markers age, tumor size, lymph node status, the histological markers tumor type or tumor grade, and TP53 mutational status in a biological sample obtained from said individual.

7. An isolated polynucleotide comprising a nucleic acid sequence encoding a polypeptide that specifically binds to an exposed epitope on Wrap53, wherein said epitope comprises at least part of position 483-496 or 449-463 of SEQ ID NO.1 and said polypeptide is selected from the group consisting of a Fab fragment, an Fv fragment, a single chain antibody, a single domain antibody and an antibody.

8. An isolated polypeptide that specifically binds to an exposed epitope on Wrap53, wherein said epitope comprises at least part of position 483-496 or 449-463 of SEQ ID NO.1 and said polypeptide is selected from the group consisting of a Fab fragment, an Fv fragment, a single chain antibody, a single domain antibody and an antibody.

9. The isolated polynucleotide of claim 7 further comprising a promoter for directing expression of said isolated polynucleotide in cells.

10. A kit for indicating a breast cancer prognosis or determining the course of a breast cancer comprising:
(a) an antibody against Wrap53 position 483-496 or 449-463 of SEQ ID NO.1; and
(b) an instruction manual explaining how to carry out at least one of the methods according to the preceding claims.

11. A method of determining the predilection for survival for an individual having breast cancer comprising:
obtaining a breast tissue sample from said individual,
measuring the amount of Wrap53 in the nucleus of cells in said breast tissue sample,
measuring the amount of Wrap53 in the cytoplasm of cells in said breast tissue sample,
comparing the amount Wrap53 measured in the nucleus of cells in said breast tissue sample to the amount of Wrap53 measured in the cytoplasm of cells in said breast tissue sample, and
determining the predilection for survival for said individual having breast cancer.

12. A method of evaluating a treatment protocol for an individual having breast cancer comprising:
obtaining a first breast tissue sample from an individual having breast cancer prior to initiating a treatment protocol for breast cancer,
measuring the amount of Wrap53 in the nucleus of cells in said first breast tissue sample,
measuring the amount of Wrap53 in the cytoplasm of cells in said first breast tissue sample,
comparing the amount Wrap53 measured in the nucleus of cells in said first breast tissue sample to the amount of Wrap53 measured in the cytoplasm of cells in said first breast tissue sample,
obtaining a second breast tissue sample from said individual after initiation or completion of said treatment protocol for breast cancer,
measuring the amount of Wrap53 in the nucleus of cells in said second breast tissue sample,
measuring the amount of Wrap53 in the cytoplasm of cells in said second breast tissue sample,
comparing the amount Wrap53 measured in the nucleus of cells in said second breast tissue sample to the amount of Wrap53 measured in the cytoplasm of cells in said second breast tissue sample, and
determining the progress of said treatment protocol by comparing the amounts of Wrap53 measured in the nucleus and/or cytoplasm of cells in said second breast tissue sample to the amount of Wrap53 measured in the nucleus and/or cytoplasm of cells in said first breast tissue sample.

13. The method of any one of claim 11, wherein the ratio of the amount of Wrap53 measured in the nucleus of cells in said breast tissue sample to the amount of Wrap53 measured in the cytoplasm of cells in said breast tissue sample is determined.

14. The method of claim 11, further comprising comparing the amount of Wrap53 measured in the nucleus or cytoplasm of the cells in said breast tissue samples or the ratio of the amount of Wrap53 measured in the nucleus of cells in said breast tissue sample to the amount of Wrap53 measured in the cytoplasm of cells in said breast tissue sample with a reference level.

15. The method of claim 11, further comprising generating a ROC curve containing values representing the amounts of Wrap53 measured in the nucleus or cytoplasm of the cells in said breast tissue samples.
